(19) United States
(12) Patent Application Publication
(10) Pub. No.: US 2026/0272336 A1
(43) Pub. Date: Sep. 17, 2026

(54) METHODS AND SYSTEMS TO OPTIMIZE GLUCOSE MONITORING USING VOICE ANALYSIS

(71) Applicant: KONINKLIJKE PHILIPS N.V., Eindhoven (NL)

(72) Inventors: Joyca Petra Wilma Lacroix, Eindhoven (NL); Aart Tijmen Van Halteren, Eindhoven (NL); Marinus Karel Johannes De Jager, Eindhoven (NL); Wilhelmus Johannes Joseph Stut, Eindhoven (NL)

(21) Appl. No.: 19/561,794

(22) Filed: Mar. 10, 2026

Related U.S. Application Data
(60) Provisional application No. 63/769,886, filed on Mar. 11, 2025.

(51) Int. Cl. A61B 5/145; A61B 5/00

(52) U.S. Cl. CPC A61B 5/14532; A61B 5/4803; A61B 5/7246; A61B 5/7267; A61B 5/746; A61B 2562/0204

(57) ABSTRACT

A method for monitoring a subject’s blood glucose level, comprising: receiving a voiceprint from the subject; correlating the received voiceprint to a determined blood glucose level for the subject, using a predefined correlation model; determining, by comparing the determined blood glucose level to a predetermined acceptable blood glucose level range for the subject, whether the determined blood glucose level is outside the predetermined acceptable blood glucose level range for the subject; and generating an alert when the determined blood glucose level is outside the predetermined acceptable blood glucose level range for the subject.

### CROSS REFERENCE TO RELATED APPLICATIONS

This patent application claims the priority benefit under 35 U.S.C. § 119(e) of U.S. Provisional Patent Application Number 63/769,886 filed March 11, 2025, the contents of which is herein incorporated by reference.

### FIELD OF THE DISCLOSURE

The present disclosure is directed generally to methods and systems for improved monitoring of a subject’s blood glucose level.

### BACKGROUND

Ambulatory monitoring and care solutions, which can include out-of-hospital and/or at-home environments or situations, often include a monitoring component where a health-related parameter is measured on a regular or continuous basis (e.g., ECG, blood glucose, etc.). These measurements are critical to: (1) make a reliable estimate of the patient’s health, and (2) intervene when needed, such as when the measurement is outside of a healthy range or shows an unhealthy pattern.

Remote diabetes management is a care solution that enables monitoring and offers support for timely controlling blood glucose (BG) levels through intervention. In the program, patients are requested to obtain glucose measurements on a regular basis. When a measurement is outside of a healthy range, a care alert is sent to a care team so that the team can reach out to the patient for education and support.

Monitoring of BG levels in a remote diabetes management program includes self-monitoring of BG levels, such as using finger-pricks to draw a blood sample. Some patients in these program use continuous blood glucose monitoring through a thin lancelet implanted subcutaneously. The first method of obtaining BG levels only provides a snapshot of the glucose level at one specific point in time, while the second method provides continuous monitoring.

Monitoring BG levels using these methods – i.e., finger-pricks or continuous BG monitoring – is considered invasive, painful, and time-consuming. It also poses a risk of potential infection and tissue damage when applied repeatedly. Unfortunately, patient non-compliance can occur for a variety of reasons. For example non-compliance is most likely when finger-pricks are used and has several causes, including being painful, time-consuming, deemed unnecessary, and/or embarrassing, among other causes. While it is less likely that patients using continuous BG monitoring will be non-compliant on a daily basis, patients may still decide to stop monitoring because of the invasive nature of continuous BG monitoring. Therefore, patient-friendly non-invasive measurement methods are an attractive alternative or complement to the traditional, more invasive, methods.

### SUMMARY OF THE DISCLOSURE

Accordingly, there is a continued need for methods and systems for more patient-friendly and non-invasive BG measurement and monitoring.

Various embodiments and implementations herein are directed to methods and systems for monitoring a subject’s blood glucose level using a non-invasive voice-based blood glucose monitoring system. The system receives a voiceprint from a subject, and correlates that voiceprint to a determined blood glucose level for the subject, using a predefined correlation model. The determined blood glucose level is compared to a predetermined acceptable blood glucose level range for the subject, and if the determined blood glucose level is outside the range, the system generates an alert or otherwise notifies the subject and/or a monitoring entity that a determined blood glucose level is outside the predetermined acceptable blood glucose level range.

Generally, in one aspect, a method for monitoring a subject’s blood glucose level is provided. The method includes: (i) receiving a voiceprint from the subject; (ii) correlating the received voiceprint to a determined blood glucose level for the subject, using a predefined correlation model; (iii) determining, by comparing the determined blood glucose level to a predetermined acceptable blood glucose level range for the subject, whether the determined blood glucose level is outside the predetermined acceptable blood glucose level range for the subject; and (iv) generating an alert when the determined blood glucose level is outside the predetermined acceptable blood glucose level range for the subject.

In accordance with an embodiment, the method further includes prompting the subject to provide the voiceprint.

In accordance with an embodiment, the predefined correlation model is a deterministic rules-based model.

In accordance with an embodiment, the predefined correlation model is a trained machine learning model. The trained machine learning model is trained with at least one of: (1) a plurality of population-level measurements of blood glucose levels and corresponding voiceprints; and (2) a plurality of subject-specific measurements of blood glucose levels and corresponding voiceprints.

In accordance with an embodiment, the method further includes generating the predefined correlation model, comprising: (i) receiving, from the subject at a plurality of different timepoints, a voiceprint and an associated blood glucose level, wherein the voiceprint and the associated blood glucose level are obtained at approximately the same time, and wherein the associated blood glucose level is obtained by a glucose monitor; (ii) extracting one or more features from each of the received voiceprints; (iii) generating a training dataset comprising the extracted one or more features from each of the received voiceprints and the associated blood glucose levels obtained by the glucose monitor; (iv) training the predefined correlation model, using the generated training dataset, to determine a blood glucose level for a subject using a received voiceprint; and (v) storing the trained predefined correlation model in memory.

In accordance with an embodiment, the method further includes extracting one or more features from the voiceprint received from the subject.

In accordance with an embodiment, the one or more extracted features comprise one or more of fundamental frequency and variations thereof, pitch, jitter, shimmer, formant frequency, and harmonic-to-noise ratio.

In accordance with an embodiment, the voiceprint received from the subject is recorded by the subject, or is provided via a live telecommunications connection.

In accordance with an embodiment, the subject is prompted to provide the voiceprint after a blood glucose level obtained from the subject, using a blood glucose monitor, is determined to be outside the predetermined acceptable blood glucose level range for the subject.

In accordance with an embodiment, the method further includes receiving one or more contextual inputs from the subject, wherein the predefined correlation model further utilizes the received one or more contextual inputs from the subject to correlate the received voiceprint to a determined blood glucose level for the subject.

In accordance with an embodiment, the one or more contextual inputs comprise one or more of an emotional state of the subject, a psychological state of the subject, health of the subject, an activity of the subject, eating or drinking information, and sleep quality.

In accordance with an embodiment, the method further includes: (i) analyzing, with a trained compliance model, information about the subject to determine whether the subject is non-compliant or likely to be non-compliant with regard to providing voiceprints and/or blood glucose levels; and (ii) prompting, when the trained compliance model determines that the subject is non-compliant or likely to be non-compliant, the subject to provide the voiceprint.

According to another aspect is a system for monitoring a subject’s blood glucose level. The system includes a predefined correlation model configured to correlate a voiceprint received from the subject to a determined blood glucose level for the subject; and a processor configured to: (i) receive a voiceprint from the subject; (ii) correlate the received voiceprint to a determined blood glucose level for the subject, using the predefined correlation model; (iii) determine, by comparing the determined blood glucose level to a predetermined acceptable blood glucose level range for the subject, whether the determined blood glucose level is outside the predetermined acceptable blood glucose level range for the subject; and (iv) generate an alert when the determined blood glucose level is outside the predetermined acceptable blood glucose level range for the subject.

In accordance with an embodiment, the system further includes a blood glucose monitor configured to obtain one or more blood glucose levels from the subject; and an audio sensor configured to obtain the voiceprint from the subject.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. The figures showing features and ways of implementing various embodiments and are not to be construed as being limiting to other possible embodiments falling within the scope of the attached claims. Also, the drawings are not necessarily to scale, emphasis instead generally being placed upon illustrating the principles of the various embodiments.

FIG. 1A is a flowchart of a method for monitoring a subject’s blood glucose level, in accordance with an embodiment.

FIG. 1B is a flowchart of a method for monitoring a subject’s blood glucose level, in accordance with an embodiment.

FIG. 2 is a schematic representation of a non-invasive voice-based blood glucose monitoring system, in accordance with an embodiment.

FIG. 3 is a flowchart of a method for training a predefined correlation model, in accordance with an embodiment.

FIG. 4 is a flowchart of a method for training a compliance model, in accordance with an embodiment.

FIG. 5 is a schematic representation of a non-invasive voice-based blood glucose monitoring system, in accordance with an embodiment.

FIG. 6 is a schematic representation of a non-invasive voice-based blood glucose monitoring system, in accordance with an embodiment.

### DETAILED DESCRIPTION OF EMBODIMENTS

The present disclosure describes various embodiments of a system and method configured for monitoring a subject’s blood glucose level using a non-invasive voice-based blood glucose monitoring system. More generally, Applicant has recognized and appreciated that it would be beneficial to provide methods and systems for more patient-friendly and non-invasive BG measurement and monitoring. Accordingly, a blood glucose monitoring system receives a voiceprint from a subject, and correlates that voiceprint to a determined blood glucose level for the subject, using a predefined correlation model. The determined blood glucose level is compared to a predetermined acceptable blood glucose level range for the subject, and if the determined blood glucose level is outside the range, the system generates an alert or otherwise notifies the subject and/or a monitoring entity that a determined blood glucose level is outside the predetermined acceptable blood glucose level range.

Advancements in the domain of voice analysis demonstrate that various diseases and pathological conditions impact a subject’s voice because they have an impact on the speech producing organs and/or the brain’s speech control system. Based on recent research, this is true for BG levels, in which variations in blood glucose are reflected in the voice. There are potentially two basic mechanisms by which BG affects changes in the voice that are detectable by computational voice analysis: changes in the elasticity of the vocal folds and anxiety. Using non-invasive methods for BG monitoring, such as using voice to estimate BG levels, may improve patient compliance and BG monitoring in general, including improving the patient experience and the burden caused by BG monitoring.

Therefore, according to an embodiment, the methods and systems described or otherwise envisioned herein optimize glucose monitoring, including ambulatory glucose monitoring. The system learns to estimate BG levels from voice samples of the individual user – optionally collected through a smartphone or computer application - at opportune moments such as when the patient makes a finger-prick measurement. The system can utilize the voice-based BG estimations when the patient is not able or willing to take an invasive BG measurement (i.e., the patient is non-compliant) and as an extra check when the invasive BG measurement is outside a healthy range, and can trigger a care alert that a monitoring entity should intervene.

According to an embodiment, the systems and methods described or otherwise envisioned herein can, in some non-limiting embodiments, be implemented as a component of or an extension of a blood glucose monitoring system, such as the Philips® Ambulatory Monitoring and Diagnostics (“AM&D”) platforms, including the AM&D ECG and AM&D Ambulatory Virtual Care Management (“AVC”) Solutions (available from Koninklijke Philips NV, the Netherlands), or as an element for a commercial product for blood glucose monitoring, either ambulatory or non-ambulatory. However, the disclosure is not limited to these devices or systems, and thus disclosure and embodiments disclosed herein can encompass any system that may utilize or benefit from blood glucose monitoring.

According to an embodiment, the methods and systems described or otherwise envisioned herein provide numerous improvements over existing blood glucose measurement systems. For example, the methods and systems reduce the risk of missing measurement values due to patient non-compliance, by introducing non-invasive voice-based measurements when the patient is not able or willing to make a traditional measurement. Studies – particularly of ambulatory virtual care services – suggest that a substantial percentage of patients are “non-compliant,” meaning that they fail to upload their measurements on a regular basis. Some patients never even start the service. This leads to missing data, poor health outcomes, and lost revenue.

According to an embodiment, the methods and systems described or otherwise envisioned herein reduce the likelihood of false positive care alerts that require human intervention, by introducing a complementary non-invasive assessment through voice, preferably close in time to the measurement that triggered a care alert. The FDA’s recommendation for fingerstick glucose meters mentions that devices should deliver measurements that are: (1) within +/- 15 percent of a highly precise lab measurement, 95 percent of the time; and (2) within +/- 20 percent of a highly precise lab measurement, 99 percent of the time. This accepted deviation from precise lab measurements means that if a blood sugar meter tells one that they’re at 120 mg/dL, most of the time that means that one could really be anywhere between 102 and 138. Moreover, the higher one’s blood sugar, the bigger the range gets. If a meter reports 200 mg/dL, the subject may be anywhere from 170 to 230, a span of 60 mg/dL. That range is substantial and the device seems even less precise when considering that as many as one in twenty measurements will fall outside of that range. In current diabetes programs, BG levels outside of the healthy range trigger a care alert at a coach team, who will intervene by reaching out to the patient within a short time frame to understand the medical situation and provide education and support to bring the BG level back to healthy value. However, considering the accepted deviations in measurements, a subset of these coach interventions are not necessary and could be avoided, thereby lowering the costs and the coach workload. and allowing true positive care alerts to be picked up within a shorter time frame. Reduction of non-compliance enables a care provider to provide a better service to patients and directly impacts revenue by increasing billability. Reduction of false positive care alerts can lower the workload of the coach team and thereby lower coach costs.

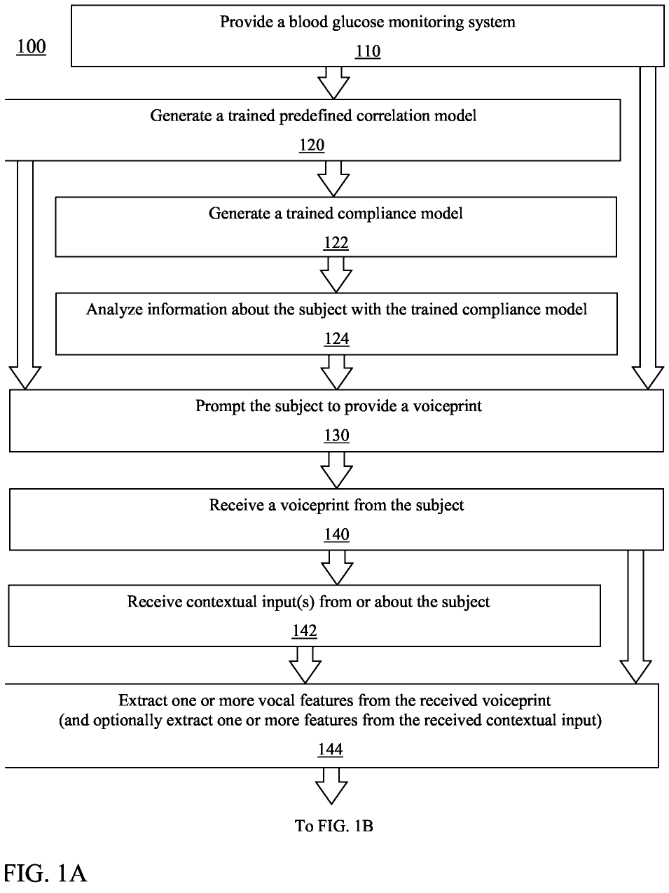

Referring to FIG. 1, in one embodiment, is a flowchart of a method 100 for blood glucose monitoring using a non-invasive voice-based blood glucose monitoring system. The methods described in connection with the figures are provided as examples only, and shall be understood to not limit the scope of the disclosure. The blood glucose monitoring system can be any of the systems described or otherwise envisioned herein. The blood glucose monitoring system can be a single system or multiple different systems.

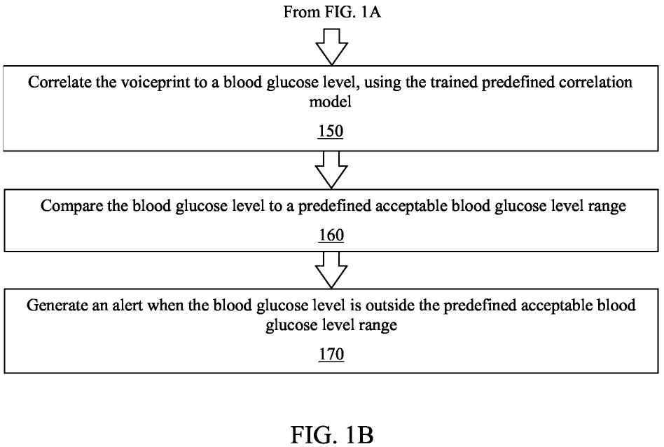

At step 110 of the method, a blood glucose monitoring system 200 is provided. Referring to an embodiment of a blood glucose monitoring system 200 as depicted in FIG. 2, for example, the system comprises one or more of a processor 220, memory 230, user interface 240, communications interface 250, and storage 260, interconnected via one or more system buses 212. It will be understood that FIG. 2 constitutes, in some respects, an abstraction and that the actual organization of the components of the system 200 may be different and more complex than illustrated. Additionally, blood glucose monitoring system 200 can be any of the systems described or otherwise envisioned herein. Other elements and components of blood glucose monitoring system 200 are disclosed and/or envisioned elsewhere herein.

According to an embodiment, the blood glucose monitoring system 200 is or comprises or is in local and/or remote communication with an ambulatory monitoring system 270. The ambulatory monitoring system 270 can be any ambulatory monitoring system that comprises or is in communication with a remote BG monitoring platform or component. Examples of an ambulatory monitoring system include the Philips Ambulatory Monitoring and Diagnostics (AM&D) Virtual Care Management (“AVC”) system, among many other possible systems. According to an embodiment, the ambulatory monitoring system comprises one or both of an invasive BG monitoring system or device (such as a lancet or continuous BG monitor) and a non-invasive BG monitoring system or device (such as a voice-based component or system as described or otherwise envisioned herein).

According to an embodiment, the blood glucose monitoring system 200 is or comprises or is in local and/or remote communication with a voiceprint and BG measurement database 280 as described or otherwise envisioned herein. According to an embodiment, the voiceprint and BG measurement database 280 comprises a plurality of voiceprints and BG measurements for one or a plurality of subjects, where each of the voiceprints and BG measurements were obtained approximately simultaneously in order to generate correlations between voiceprints and BG measurements for a subject. For example, a subject may provide a plurality of voiceprints and BG measurements - obtained approximately simultaneously (where “approximately simultaneously” typically means as close in time as possible but preferably within seconds or minutes of each other) – over the course of an initial time period such as a day, two or more days, a week, two or more weeks, a month, or two or more months. Voiceprints and BG measurements can be obtained during the initial time period at one or numerous points throughout each day, such as around (e.g., before, during, or after) mealtimes, waking times, bedtimes, rest times, active times, and/or other times. The number of voiceprints and BG measurements obtained from a subject can depend on a wide variety of factors, including but not limited to the quality of the voiceprints and BG measurements, physiological or demographic parameters of the subject, parameters of a correlation model that is trained with the stored voiceprints and BG measurements, and/or many other factors.

As described or otherwise envisioned herein, the blood glucose monitoring system 200 comprises or is in remote and/or local communication with a predefined correlation model that receives as input a voiceprint from a subject, correlates that received voiceprint to a BG measurement for the subject, and outputs the determined BG measurement for the subject. As described or otherwise envisioned herein, the predefined correlation model can receive and utilize additional input to correlate that input to a BG measurement for the subject.

According to an embodiment, the predefined correlation model is a deterministic rules-based model, such that it is a logical framework that specifies relationships or identifies correlations between variables in a system using a set of deterministic rules. The predefined correlation model can implement the deterministic rules to correlate a received voiceprint to a BG measurement for a subject. The rules for the predefined correlation model can be experimentally derived based on subject-level and/or population-level data comprising voiceprints and associated BG measurements. The rules for the predefined correlation model can be pre-programmed or otherwise provided to or received by the system.

According to another embodiment, the predefined correlation model is a trained machine learning model. The machine learning model can be a neural network, a CNN, a classifier, a random forest model, or any other trained machine learning model. The predefined correlation model can be trained, for example, to correlate a voiceprint received from a subject to a BG measurement for that subject, among other possible inputs and outcomes.

The predefined correlation model can be any model that can be trained to utilize the input to generate the output, as described or otherwise envisioned herein. For example, the predefined correlation model can be a neural network or other trained machine learning model. Thus, according to an embodiment, the BG monitoring system comprises a trained predefined correlation model that receives the input data (e.g., a voiceprint from a subject) and outputs data regarding a BG measurement for the subject.

The predefined correlation model can be trained in a variety of different ways. According to one embodiment, the model is trained with at least one of: (1) a plurality of population-level measurements of blood glucose levels and corresponding voiceprints; and (2) a plurality of subject-specific measurements of blood glucose levels and corresponding voiceprints. According to one embodiment, the predefined correlation model is trained in an unsupervised manner or in a supervised manner.

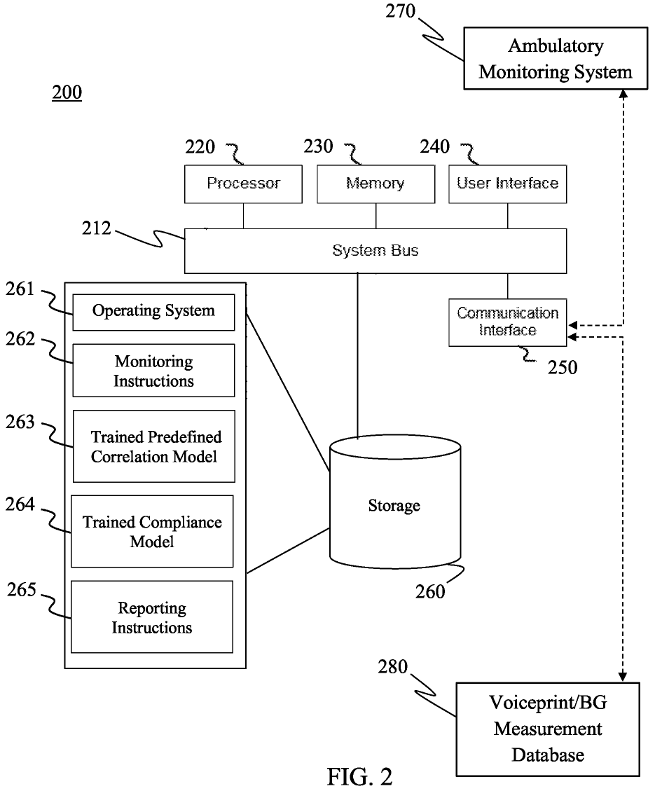

Accordingly, at step 120 of the method, the predefined correlation model is generated. Referring to FIG. 3, in one embodiment, is a flowchart of a method 300 for training the predefined correlation model of the blood glucose monitoring system 200. This method may be performed by the blood glucose monitoring system or may be performed by another system such as a specialized machine learning model training system.

At step 310 of method 300, the training system receives training data which will be used to train the model. The training data can be any data sufficient to train the model to utilize the described input data to generate the described output. For example, the training data may comprise at least one of: (1) a plurality of population-level measurements of blood glucose levels and corresponding voiceprints, comprising data for a plurality of different subjects; and (2) a plurality of subject-specific measurements of blood glucose levels and corresponding voiceprints, obtained from a single subject. Thus, according to an embodiment, the predefined correlation model may be a population-level model, trained with population-level training data. According to another embodiment, the predefined correlation model may be a subject-level model, trained with subject-level training data. Thus, according to one embodiment, the predefined correlation model is trained to be used for a plurality of different subjects. According to another embodiment, the predefined correlation model is trained to be used for one specific subject.

Thus, at step 310, the training system receives training data that comprises data received from a subject (or multiple subjects) at a plurality of different timepoints. The data comprises a voiceprint and an associated blood glucose level, wherein the voiceprint and the associated blood glucose level are obtained at approximately the same time, and wherein the associated blood glucose level is obtained by a glucose monitor.

This training data, which could be utilized in a supervised or unsupervised manner, can comprise 100s or 1000s of datapoints, and can optionally be updated with new datapoints. The training data may also comprise other information. This training data may be obtained and curated by an expert such as a clinician, or it may be obtained and curated under the supervision of a clinician, or it may be obtained and utilized without curation. The training data may be received from any source. For example, the training data may be received from an electronic medical record database or system, or any other component of the blood glucose monitoring system or a training system. According to an embodiment, the blood glucose monitoring system 200 comprises or is in direct or indirect communication with a database – such as database 280 – which comprises some or all of the training data set.

According to an embodiment, the training system may comprise a data pre-processor or similar component or algorithm configured to process the received training data. For example, the data pre-processor analyzes the training data to remove noise, bias, errors, and other potential issues. The data pre-processor may also analyze the input data to remove low quality data. Many other forms of data pre-processing or data point identification and/or extraction are possible.

At step 320 of the method, the training system extracts one or more vocal features from a received voiceprint(s). The vocal feature(s) can be extracted from a voiceprint as the voiceprint is received, or stored voiceprints can be analyzed in batches. The vocal feature(s) can be any feature that is useful for correlating a voiceprint from a subject to a BG measurement for the subject. Non-limiting examples of vocal feature(s) extracted from a voiceprint include fundamental frequency and variations thereof, pitch, jitter, shimmer, formant frequency, and harmonic-to-noise ratio, among others.

In embodiments where the predefined correlation model uses information other than vocal features to determine blood glucose levels, the training system can extract features from that other information. Examples of other information include contextual information, and the predefined correlation model can utilize contextual inputs from a subject to correlate a received voiceprint to a determined blood glucose level for the subject. Non-limiting examples of contextual information or inputs include an emotional state of the subject, a psychological state of the subject, health of the subject, an activity of the subject, eating or drinking information, and sleep quality, among others.

At step 330 of the method, the training system generates a training dataset from the data, comprising the extracted one or more features from each of the received voiceprints and the associated blood glucose levels obtained by the glucose monitor, and optionally including features extracted from contextual information. Once generated, the training dataset may be stored and/or may be utilized immediately.

At step 340 of the method, the training system trains the predefined correlation model, using the training data, to correlate a received voiceprint (and optionally received contextual information) with a blood glucose level for the subject. The predefined correlation model is trained using any method for training such a model. The trained predefined correlation model is a unique model based on the training data used to train the model. Following training, the system comprises a trained predefined correlation model.

Thus, following training, the predefined correlation model is a specialized model configured to receive the input (namely, a voiceprint and optionally contextual information) and generate the very specific output, namely the determined blood glucose level for the subject.

At step 350 of the method, the trained predefined correlation model is stored for future use. According to an embodiment, the trained predefined correlation model may be stored in local or remote storage.

Returning to method 100 in FIG. 1, the system can analyze information about the subject to determine whether the subject is non-compliant or likely to be non-compliant with regard to providing voiceprints and/or blood glucose levels. The system can analyze this information using a trained compliance model.

As described or otherwise envisioned herein, the blood glucose monitoring system 200 comprises or is in remote and/or local communication with a trained compliance model that receives as input information about the subject, such as demographic, historical, compliance, health, and/or other information, and correlates that input information with a likelihood that the subject will be compliant or non-compliant with regard to monitoring their blood glucose levels, and more specifically to providing voiceprints and/or blood glucose levels.

Thus, at step 122 of the method, the system can generate a trained compliance model. The trained compliance model can be a machine learning model. The machine learning model can be a neural network, a CNN, a classifier, a random forest model, or any other trained machine learning model. The trained compliance model can be trained, for example, to correlate information about a subject with a likelihood or estimate or prediction that the subject will be compliant or non-compliant with regard to monitoring their blood glucose levels.

The compliance model can be trained in a variety of different ways. According to one embodiment, the model is trained with at least one of: (1) compliance information and/or other information about a plurality of subjects; and (2) compliance information and/or other information about a single subject. According to one embodiment, the compliance model is trained in an unsupervised manner or in a supervised manner.

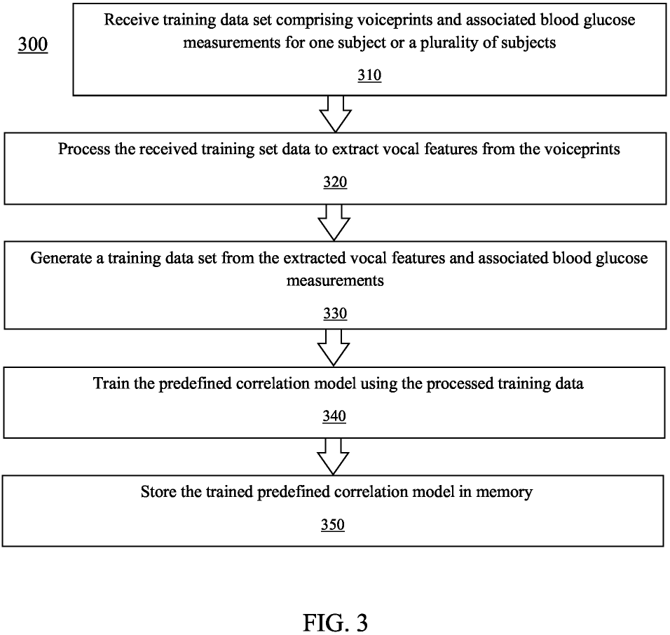

Referring to FIG. 4, in one embodiment, is a flowchart of a method 400 for training the compliance model of the blood glucose monitoring system 200. This method may be performed by the blood glucose monitoring system or may be performed by another system such as a specialized machine learning model training system.

At step 410 of method 400, the training system receives training data which will be used to train the model. The training data can be any data sufficient to train the model to utilize the described input data to generate the described output. For example, the training data may comprise at least one of: (1) compliance information and/or other information about a plurality of subjects; and (2) compliance information and/or other information about a single subject. Thus, according to an embodiment, the compliance model may be a population-level model, trained with population-level training data. According to another embodiment, the compliance model may be a subject-level model, trained with subject-level training data. Thus, according to one embodiment, the compliance model is trained to be used for a plurality of different subjects. According to another embodiment, the compliance model is trained to be used for one specific subject.

Thus, at step 410, the training system receives training data that comprises data received from a subject (or multiple subjects). The data comprises any information that could be relevant to compliance, including but not limited to demographic, historical, compliance, health, and/or other information.

This training data, which could be utilized in a supervised or unsupervised manner, can comprise 100s or 1000s of datapoints, and can optionally be updated with new datapoints. The training data may also comprise other information. This training data may be obtained and curated by an expert such as a clinician, or it may be obtained and curated under the supervision of a clinician, or it may be obtained and utilized without curation. The training data may be received from any source. For example, the training data may be received from an electronic medical record database or system, or any other component of the blood glucose monitoring system or a training system. According to an embodiment, the blood glucose monitoring system 200 comprises or is in direct or indirect communication with a database – such as database 280 – which comprises some or all of the training data set.

According to an embodiment, the training system may comprise a data pre-processor or similar component or algorithm configured to process the received training data. For example, the data pre-processor analyzes the training data to remove noise, bias, errors, and other potential issues. The data pre-processor may also analyze the input data to remove low quality data. Many other forms of data pre-processing or data point identification and/or extraction are possible.

At step 420 of the method, the training system extracts one or more features from the received training data. The feature(s) can be any feature that is useful for correlating information about the subject with a likelihood or estimate or prediction that the subject will be compliant or non-compliant with regard to monitoring their blood glucose levels.

At step 430 of the method, the training system generates a training dataset from the data, comprising the extracted one or more features from each of the received information. Once generated, the training dataset may be stored and/or may be utilized immediately.

At step 440 of the method, the training system trains the compliance model, using the training data, to correlate compliance information about the subject with a likelihood or estimate or prediction that the subject will be compliant or non-compliant with regard to monitoring their blood glucose levels. The compliance model is trained using any method for training such a model. The trained compliance ion model is a unique model based on the training data used to train the model. Following training, the system comprises a trained compliance model.

Thus, following training, the compliance model is a specialized model configured to receive the input (namely, compliance information or other information about the subject) and generate the very specific output, namely the likelihood or estimate or prediction that the subject will be compliant or non-compliant with regard to monitoring their blood glucose levels.

At step 450 of the method, the trained compliance model is stored for future use. According to an embodiment, the trained compliance model may be stored in local or remote storage.

Returning to method 100 in FIG. 1, at step 124 of the method, the system analyzes – with the trained compliance model – information about the subject to determine a likelihood or estimate or prediction that the subject will be compliant or non-compliant with regard to monitoring their blood glucose levels (such as providing voiceprints and/or blood glucose levels). The information about the subject can include compliance information, such as demographic, historical, compliance, health, and/or other information.

Thus, following step 124 of the method, the system comprises a likelihood or estimate or prediction that the subject will be compliant or non-compliant with regard to monitoring their blood glucose levels. According to an embodiment, the system determines that the subject is likely to be non-compliant with regard to monitoring their blood glucose levels, and thus progresses to step 130 of the method.

At step 130 of the method, the blood glucose monitoring system 200 prompts the subject to provide a voiceprint. This prompt can be triggered in response to a variety of different factors. As described above, the prompt can be triggered by a determination that the subject is likely to be non-compliant with regard to monitoring their blood glucose levels. According to another embodiment, the prompt can be triggered in response to a timer or other predetermined schedule or program. According to another embodiment, the prompt can be triggered in response to determining that an obtained blood glucose measurement (or other biometric) is outside a predetermined acceptable blood glucose level range for the subject. According to another embodiment, the prompt can be triggered in response to an action or activity taken by the subject, such as eating a meal, sleeping, physical activity, and/or other action or activity.

The prompt can be provided to the subject in a wide variety of ways. According to an embodiment, the subject is remote to one or more components of the blood glucose monitoring system 200, and a component of the system communicates a prompt to the subject. For example, the subject can interface with a user interface through which a prompt to obtain a voiceprint is provided. The user interface can be any user interface that a subject can use, including but not limited to a smartphone (such as a smartphone app), a desktop, laptop, tablet, smartwatch, smart glasses, other wearable device, television, speaker, and/or any other user interface. According to an embodiment, the prompt can be an instruction such as “please provide a voiceprint,” or it can be a haptic notification, a sound, a visible instruction (e.g., light, displayed text, pattern), and/or any other method of providing a prompt to obtain a voiceprint.

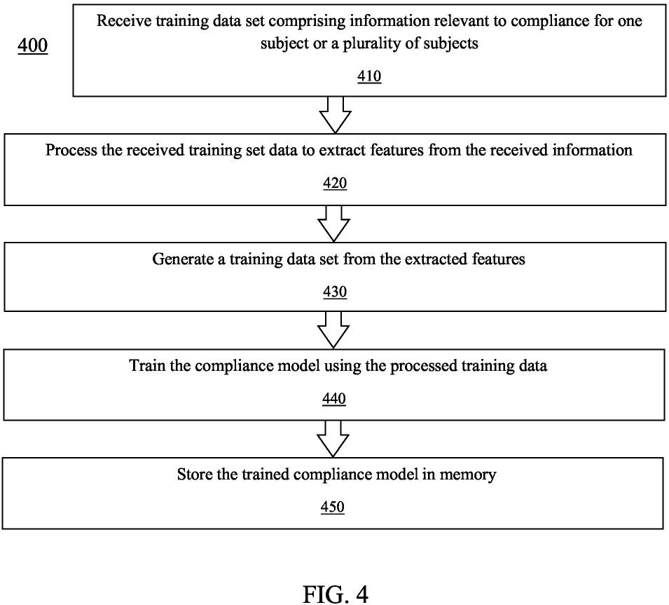

Referring to FIG. 5, in one embodiment, is a schematic representation of a blood glucose monitoring environment 500, including a subject 510 and a blood glucose monitoring system that comprises one or more of a mobile application 520, remote components 530, and monitoring components 540. According to this non-limiting example, the blood glucose monitoring system learns to estimate BG levels from voice samples of the individual user that are collected through a smartphone app 520 at opportune moments (e.g., when the patient makes a finger-prick measurement). Subsequently, the system uses the voice-based BG estimations when the patient is not able or willing to take an invasive BG measurement and/or as an extra check when the invasive BG measurement is out of the healthy range.

According to this embodiment, blood glucose monitoring environment 500 comprises a smartphone mobile application 520 that interfaces with the patient 510 to collect a voiceprint when an invasive BG measurement is taken using glucose meter 522, and prompts the patient to collect a voiceprint when, for example: (1) the patient is not willing or able to obtain an invasive BG measurement; and/or (2) the invasive BG measurement falls outside of a healthy range and a care alert is triggered.

According to this embodiment, blood glucose monitoring environment 500 comprises remote components 530, such as cloud-based components. These components can include, for example, database(s) that store voiceprints and/or BG measurements, and an AI algorithm trained to predict BG levels from voiceprints. This AI can be trained (or fine-tuned) at regular intervals when new voiceprints and related BG measurements are provided.

According to this embodiment, blood glucose monitoring environment 500 comprises monitoring components 540. The monitoring components 540 can include, for example, a coach that monitors care alerts and non-compliance cases. The coach can monitor multiple patients. A care alert indicates that a BG level for a specific patient is outside (too high or too low) a predetermined BG range or interval. Non-compliance cases indicate that a specific patient is at risk of providing insufficient BG measurements to remain eligible to use the service. The monitoring components 540 can further include, for example, a non-compliance prediction AI that identifies when patients are likely to become non-compliant. This AI can be trained on historic data from the patient population.

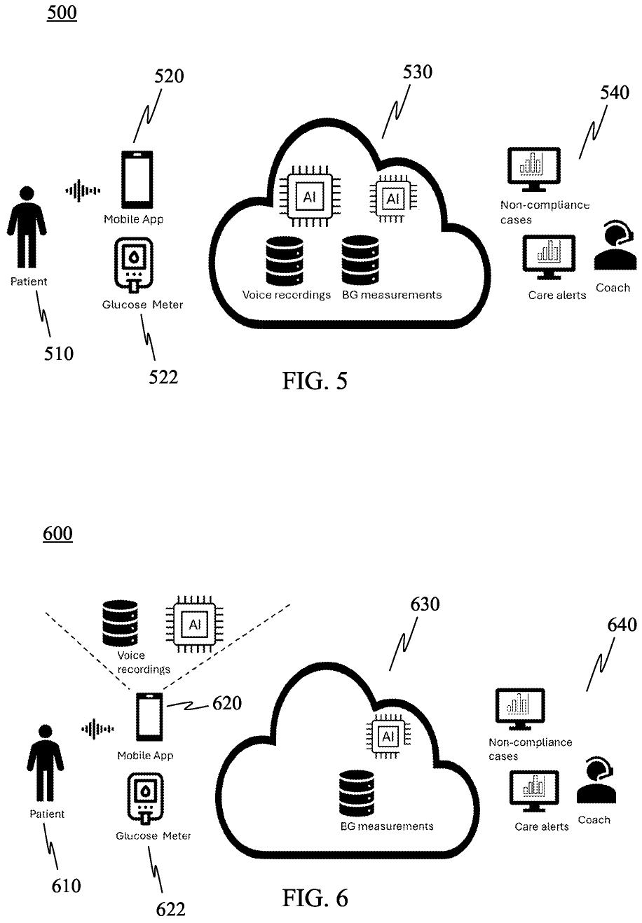

Referring to FIG. 6, in another embodiment, is a schematic representation of a blood glucose monitoring environment 600, including a subject 610 and a blood glucose monitoring system that comprises one or more of a mobile application 620, remote components 630, and monitoring components 640. According to this non-limiting example, the blood glucose monitoring system learns to estimate BG levels from voice samples of the individual user that are collected through a smartphone app 620 at opportune moments (e.g., when the patient makes a finger-prick measurement). Subsequently, the system uses the voice-based BG estimations when the patient is not able or willing to take an invasive BG measurement and/or as an extra check when the invasive BG measurement is out of the healthy range.

According to this embodiment, blood glucose monitoring environment 600 comprises a smartphone mobile application 620 that interfaces with the patient 610 to collect a voiceprint when an invasive BG measurement is taken using glucose meter 622, and prompts the patient to collect a voiceprint when, for example: (1) the patient is not willing or able to obtain an invasive BG measurement; and/or (2) the invasive BG measurement falls outside of a healthy range and a care alert is triggered. In this embodiment, smartphone 620 comprises a database of voiceprints, and comprises an AI algorithm trained to predict BG levels from voiceprints. This AI can be trained (or fine-tuned) at regular intervals when new voiceprints and related BG measurements are provided.

According to this embodiment, blood glucose monitoring environment 600 comprises remote components 630, such as cloud-based components. These components can include, for example, a database that stores BG measurements.

According to this embodiment, blood glucose monitoring environment 600 comprises monitoring components 640. The monitoring components 640 can include, for example, a coach that monitors care alerts and non-compliance cases. The coach can monitor multiple patients. A care alert indicates that a BG level for a specific patient is outside (too high or too low) a predetermined BG range or interval. Non-compliance cases indicate that a specific patient is at risk of providing insufficient BG measurements to remain eligible to use the service. The monitoring components 640 can further include, for example, a non-compliance prediction AI that identifies when patients are likely to become non-compliant. This AI can be trained on historic data from the patient population.

Returning to method 100 in FIG. 1, at step 130 of the method, the blood glucose monitoring system 200 prompts the subject to provide a voiceprint. Referring to FIGS. 5 and 6 as examples, the prompt can be provided to the patient via a smartphone, such as via a smartphone app that causes an alert or notification to be provided to the patient. This notification can include a haptic notification, a sound, a visible notification, and/or any other notification. According to an embodiment, the smartphone provides the notification to a wearable worn by the patient, and/or to another user interface in the vicinity of the patient, such as a smart speaker, television, smart mirror, or other user interface.

The voiceprint can be provided to the system in a wide variety of ways. Referring to FIGS. 5 and 6 as examples, the voiceprint can be provided to the system via the smartphone and smartphone app 620. The smartphone can record a voiceprint, and can store it and/or transmit it to another component of the system for subsequent use by the system. The voiceprint can alternatively be provided via any user interface that comprises an audio sensor, such as a smart watch, smart glasses, smart speaker, television, smart mirror, desktop, laptop, tablet, and/or any other user interface that can comprise an audio sensor. Once obtained, the voiceprint can be stored in local and/or remote memory, and/or it can be utilized immediately by the system.

At step 140 of the method, the system receives the voiceprint from the subject. As described above, the voiceprint can be provided in a wide variety of ways. According to an embodiment, the voiceprint is obtained remotely via a user interface, such as a smartphone audio sensor, and is transmitted to one or more other components of the system. According to another embodiment, the voiceprint is obtained via a live telecommunications connection, such as via an audio call or video call. Once received, the voiceprint can be stored in local and/or remote memory, and/or it can be utilized immediately by the system.

The voiceprint can be a predetermined word or words, phrase, text, sound, noise, or any other vocal message. For example, the voiceprint may be a sentence that the subject repeats or announces each time, or may vary from time to time. The optimal vocal message for the voiceprint may be universal for all subjects, or may be specific to a specific subject.

At optional step 142 of the method, the blood glucose monitoring system 200 receives one or more contextual inputs from and/or about the subject. The contextual inputs can be received from the subject directly, such as through a user interface (e.g., a smartphone, smart watch, smart glasses, smart speaker, television, smart mirror, desktop, laptop, tablet, and/or any other user interface). The contextual inputs can be received from a database of contextual information, wherein the database can be a remote and/or local database, and can be a component of the system or alternatively the system can be in local and/or remote communication with the database of contextual information. The contextual inputs/information can be any information that the predefined correlation model can utilize to correlate the voiceprint with a BG level for the subject. As non-limiting examples, the contextual inputs/information can be one or more of an emotional state of the subject, a psychological state of the subject, health of the subject, an activity of the subject, eating or drinking information, and sleep quality. Many other contextual inputs/information are possible. Once received, the contextual inputs/information can be utilized immediately, and/or it can be stored in local or remote memory for future use by the system.

At step 144 of the method, the blood glucose monitoring system 200 extracts one or more vocal features from the voiceprint received from the subject. The vocal features can be extracted from the voiceprint using known methods for extracting features. The vocal feature(s) can be extracted from a voiceprint as the voiceprint is received, or stored voiceprints can be analyzed in batches. The vocal feature(s) can be any feature that is useful for correlating a voiceprint from a subject to a BG measurement for the subject. Non-limiting examples of vocal feature(s) extracted from a voiceprint include fundamental frequency and variations thereof, pitch, jitter, shimmer, formant frequency, and harmonic-to-noise ratio, among others.

In embodiments where the predefined correlation model uses contextual inputs/information, the system can extract contextual features from that received contextual inputs/information. The contextual features can be extracted from the received contextual inputs/information using known methods for extracting features. The feature(s) can be extracted from contextual inputs/information as it is received, or stored contextual inputs/information can be analyzed in batches.

Once the vocal features are extracted (and optionally the contextual information features are extracted), they can be utilized immediately, and/or they can be stored in local or remote memory for future use by the system.

At step 150 of the method, the blood glucose monitoring system 200 correlates the received voiceprint to a determined blood glucose level for the subject, using the predefined correlation model. According to an embodiment, the extracted vocal features are provided to the predefined correlation model, optionally with the extracted contextual input features, and the predefined correlation model analyzes the features to determine, estimate, or predict the blood glucose level for the subject. The predefined correlation model correlates the features to the blood glucose level for the subject as described or otherwise envisioned herein.

Once the blood glucose level for the subject is determined, estimated, or predicted, it can be utilized immediately, and/or it can be stored in local or remote memory for future use by the system.

At step 160 of the method, the blood glucose monitoring system 200 compares the determined blood glucose level, output from the predefined correlation model based on the input (including the extracted vocal features and optionally the extracted contextual information features), to a predetermined acceptable blood glucose level range for the subject. The predetermined acceptable blood glucose level range for the subject can be preprogrammed, experimentally derived, based on healthcare standards, or otherwise determined. This range can be provided to the system by a clinician, preprogrammed into the system, or otherwise provided to the system.

Based on the comparison, the system determines whether the determined blood glucose level is without or outside the predetermined acceptable blood glucose level range for the subject. According to an embodiment, for example, the expected value for blood glucose for an adult male may be between 70mg/dL (3.9 mmol/L) and 100mg/dL (5.6 mmol/L), although of course variations are possible. As a non-limiting example, if the predefined correlation model determines that the blood glucose level is 110mg/dL, this falls outside the expected range for the adult male.

At step 170 of the method, the blood glucose monitoring system 200 determines that the blood glucose level is outside the predetermined acceptable blood glucose level range for the subject (for example, the level is below the range or the level is above the range, or the level otherwise violates a rule or triggers an alert) and generates an alert indicating that the blood glucose level is outside the predetermined acceptable blood glucose level range for the subject. The alert can be generated locally to the subject, and/or the alert can be generated by the system remote from the subject.

The alert can be any alert or notification that the determined blood glucose level is outside the predetermined acceptable blood glucose level range for the subject. The alert may be provided to a care provider, such as a monitoring entity (which may be a component of the system, or a human healthcare provider), via a user interface. The alert can be an audible alert, a visible alert, a haptic alert, a text, and/or any other notification. According to an embodiment, the alert can be provided via any user interface, including but not limited to a smart watch, smart glasses, smart speaker, television, smart mirror, desktop, laptop, tablet, and/or any other user interface.

Therefore, the systems and methods described or otherwise envisioned herein comprise a predefined correlation model that analyzes a voiceprint from a user in order to determine, estimate, or predict the user’s blood glucose level.

According to an embodiment, in order to predict BG levels from voice recordings, the predefined correlation model is trained with voice recordings of an individual. Training data comes from a voice recordings database and a BG measurements database (which can be one or multiple databases). To overcome the cold start problem (i.e., when there are insufficient voice recordings of a patient available for a reliable BG prediction) the predefined correlation model can be initially trained on population data, but can be trained on patient-specific recordings as they become available.

According to an embodiment, the compliance model uses a plurality of features to determine whether a patient is at risk of uploading insufficient BG measurements. Features can include patient characteristics (such as age, use of medication, etc.) and observed patient behavior (e.g. number of days since last upload, time on service, etc.). This algorithm is trained on the data of a representative sample from the patient population. The algorithm may only be retrained occasionally (e.g., quarterly). Non-compliance risk can be calculated for a patient at a regular interval (e.g., daily).

If a patient has not provided BG measurements for a while and the non-compliance risk exceeds a predetermined threshold, the mobile app can be triggered to interact with the user to collect a recent voice recording. This voice recording can then be used to determine, estimate, or predict a BG measurement. This estimated BG measurement could trigger a care alert on the coach dashboard, as described herein. In an alternative embodiment the voice recording is obtained via a telephone conversation that the coach has with the patient. This voice recording can then be used to predict a BG value, which can then trigger a care alert.

In an extended embodiment, the system can include a training period (for the predefined correlation model) to optimize BG estimation based on voice, where a patient submits standardized voice samples and BG measurements at regular moments during the day for a predefined period. Standardization of voice samples can be achieved by predefining the voice samples in terms of a few parameters that have been shown to generate strong correlations between voice and BG, e.g. including certain sounds (e.g., ‘aaa’ and ‘oooo’), words (e.g., longer or shorter words) or loudness of speech (e.g., asking for louder voice sample). These parameters can be learned from all collected voice sample-BG pairs or from literature when available.

In an alternative embodiment, additional contextual input is collected. The contextual information/input may be known to impact BG levels (e.g., activity, last eating moment, sleep quality) when voice and BG samples are collected, to further optimize the accuracy and certainty of the BG predictions. This can be done effectively by collecting voice samples under prescribed conditions in a training period (e.g., while fasting, before lunch, after intense activity, etc.). The contextual input collected at the moment of a voice sample can serve as additional input to the predefined correlation model to improve prediction accuracy and certainty.

According to an embodiment, one way to collect contextual input for training the system is to connect the collection of voice samples to the Glucose Day Curve (“GDC”) that is prescribed to patients in certain situations. A GDC measures glucose over the course of a day. There are different GDCs depending on the number of measurement points. The following are examples of GDCs, although many other are possible: (1) a 2-point GDC, including fasting and before dinner; (2) a 4-point GDC, including fasting, before lunch, before dinner, and before sleeping; and (3) a 5-point GDC, including fasting, 90 minutes after breakfast, 90 minutes after lunch, 90 minutes after dinner, and before sleeping. Many variations and alternatives are possible.

According to an embodiment, the subject’s conditions can be recorded as they naturally occur at the moment of a voice sample and BG sample (e.g., without prescribing them) and used for training the system. The patient could be asked to read out loud the value measured by the glucose monitor (e.g., speaking “one hundred twenty” when the monitor shows 120mg/dL). This is not only useful for training the algorithm, but also creates awareness among patients.

Referring to FIG. 2 is a schematic representation of a blood glucose monitoring system 200. System 200 may be any of the systems described or otherwise envisioned herein and may comprise any of the components described or otherwise envisioned herein. It will be understood that FIG. 2 constitutes, in some respects, an abstraction and that the actual organization of the components of the system 200 may be different and more complex than illustrated.

According to an embodiment, system 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system or may located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

According to an embodiment, storage 260 of system 200 may store one or more algorithms, modules, and/or instructions to carry out one or more functions or steps of the methods described or otherwise envisioned herein. For example, the system may comprise, among other instructions or data, monitoring instructions 262, a trained predefined correlation model 263, a trained compliance model 264, and/or reporting instructions 265.

According to an embodiment, monitoring instructions 262 direct the system to monitor the subject. For example, the monitoring instructions 262 can direct the system to analyze – with the trained compliance model – information about the subject to determine a likelihood or estimate or prediction that the subject will be compliant or non-compliant with regard to monitoring their blood glucose levels (such as providing voiceprints and/or blood glucose levels). The monitoring instructions 262 can also direct the system to prompt the subject to provide a voiceprint (for training and/or for post-training analysis). This prompt can be triggered in response to a variety of different factors. As described above, the prompt can be triggered by a determination that the subject is likely to be non-compliant with regard to monitoring their blood glucose levels. According to another embodiment, the prompt can be triggered in response to a timer or other predetermined schedule or program. According to another embodiment, the prompt can be triggered in response to determining that an obtained blood glucose measurement (or other biometric) is outside a predetermined acceptable blood glucose level range for the subject. According to another embodiment, the prompt can be triggered in response to an action or activity taken by the subject, such as eating a meal, sleeping, physical activity, and/or other action or activity.

According to an embodiment, trained predefined correlation model 263 is a trained model that receives as input a voiceprint from a subject, correlates that received voiceprint to a BG measurement for the subject, and outputs the determined BG measurement for the subject. As described or otherwise envisioned herein, the predefined correlation model can receive and utilize additional input to correlate that input to a BG measurement for the subject.

According to an embodiment, the predefined correlation model is a deterministic rules-based model, such that it is a logical framework that specifies relationships or identifies correlations between variables in a system using a set of deterministic rules. According to another embodiment, the predefined correlation model is a trained machine learning model. The machine learning model can be a neural network, a CNN, a classifier, a random forest model, or any other trained machine learning model. The predefined correlation model can be trained, for example, to correlate a voiceprint received from a subject to a BG measurement for that subject, among other possible inputs and outcomes.

The predefined correlation model can be any model that can be trained to utilize the input to generate the output, as described or otherwise envisioned herein. For example, the predefined correlation model can be a neural network or other trained machine learning model. Thus, according to an embodiment, the BG monitoring system comprises a trained predefined correlation model that receives the input data (e.g., a voiceprint from a subject) and outputs data regarding a BG measurement for the subject.

According to an embodiment, trained predefined correlation model 263 is trained according to the methods and systems described or otherwise envisioned herein to analyze a voiceprint and/or contextual information. Thus, the trained predefined correlation model is configured to process many thousands or millions of datapoints in the input data used to train the algorithm, as well as to process and analyze a voiceprint and/or contextual information. For example, generating a functional and skilled trained algorithm using an automated process such as feature identification and extraction and subsequent training requires processing of millions of datapoints from input data and the generated features. This can require millions or billions of calculations to generate a novel trained comparison algorithm from those millions of datapoints and millions or billions of calculations. As a result, each trained algorithm is novel and distinct based on the input data and parameters of the machine learning algorithm, and thus improves the functioning of the blood glucose monitoring system. Thus, generating a functional and skilled trained classifier comprises a process with a volume of calculation and analysis that a human brain cannot accomplish in a lifetime, or multiple lifetimes.

According to an embodiment, trained compliance model 264 is a machine learning model. The machine learning model can be a neural network, a CNN, a classifier, a random forest model, or any other trained machine learning model. The trained compliance model can be trained, for example, to correlate information about a subject with a likelihood or estimate or prediction that the subject will be compliant or non-compliant with regard to monitoring their blood glucose levels. The compliance model can be trained in a variety of different ways. According to one embodiment, the model is trained with at least one of: (1) compliance information and/or other information about a plurality of subjects; and (2) compliance information and/or other information about a single subject. According to one embodiment, the compliance model is trained in an unsupervised manner or in a supervised manner. The system analyzes – with the trained compliance model – information about the subject to determine a likelihood or estimate or prediction that the subject will be compliant or non-compliant with regard to monitoring their blood glucose levels (such as providing voiceprints and/or blood glucose levels). The information about the subject can include compliance information, such as demographic, historical, compliance, health, and/or other information.

According to an embodiment, reporting instructions 265 direct the system to generate and provide an alert. For example, the system may determine that the blood glucose level is outside the predetermined acceptable blood glucose level range for the subject (for example, the level is below the range or the level is above the range, or the level otherwise violates a rule or triggers an alert) and generates an alert indicating that the blood glucose level is outside the predetermined acceptable blood glucose level range for the subject. The alert can be generated locally to the subject, and/or the alert can be generated by the system remote from the subject. The alert can be any alert or notification that the determined blood glucose level is outside the predetermined acceptable blood glucose level range for the subject. The alert may be provided to a care provider, such as a monitoring entity (which may be a component of the system, or a human healthcare provider), via a user interface. The alert can be an audible alert, a visible alert, a haptic alert, a text, and/or any other notification. According to an embodiment, the alert can be provided via any user interface, including but not limited to a smart watch, smart glasses, smart speaker, television, smart mirror, desktop, laptop, tablet, and/or any other user interface.

The present invention may be a system, a method, and/or a computer program product. The computer program product may include a non-transitory computer readable storage medium (or media) having computer readable program instructions thereon for causing a system or processor to carry out aspects of the present invention. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any combination of the foregoing, among other possibilities. Computer readable program instructions described herein can be downloaded to respective computing/processing devices from a computer readable storage medium or to an external computer or external storage device via a network, for example, the internet, a local area network, and/or a wireless network. Aspects of the present invention are described herein with reference to flowchart illustrations and/or block diagrams of methods, apparatus, systems, and computer program products according to embodiments of the invention. It will be understood that each block of the flowchart illustrations and/or block diagrams, and combinations of blocks in the flowchart illustrations and/or block diagrams, can be implemented by computer readable program instructions.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A method for monitoring a subject’s blood glucose level, comprising:
receiving a voiceprint from the subject;
correlating the received voiceprint to a determined blood glucose level for the subject, using a predefined correlation model;
determining, by comparing the determined blood glucose level to a predetermined acceptable blood glucose level range for the subject, whether the determined blood glucose level is outside the predetermined acceptable blood glucose level range for the subject; and
generating an alert when the determined blood glucose level is outside the predetermined acceptable blood glucose level range for the subject.

2. The method of claim 1, further comprising the step of prompting the subject to provide the voiceprint.

3. The method of claim 1, wherein the predefined correlation model is a deterministic rules-based model.

4. The method of claim 1, wherein the predefined correlation model is a trained machine learning model, the trained machine learning model trained with at least one of: (1) a plurality of population-level measurements of blood glucose levels and corresponding voiceprints; and (2) a plurality of subject-specific measurements of blood glucose levels and corresponding voiceprints.

5. The method of claim 1, further comprising the step of generating the predefined correlation model, comprising:
receiving, from the subject at a plurality of different timepoints, a voiceprint and an associated blood glucose level, wherein the voiceprint and the associated blood glucose level are obtained at approximately the same time, and wherein the associated blood glucose level is obtained by a glucose monitor;
extracting one or more features from each of the received voiceprints;
generating a training dataset comprising the extracted one or more features from each of the received voiceprints and the associated blood glucose levels obtained by the glucose monitor;
training the predefined correlation model, using the generated training dataset, to determine a blood glucose level for a subject using a received voiceprint; and
storing the trained predefined correlation model in memory.

6. The method of claim 1, further comprising the step of extracting one or more features from the voiceprint received from the subject.

7. The method of claim 6, wherein the one or more extracted features comprise one or more of fundamental frequency and variations thereof, pitch, jitter, shimmer, formant frequency, and harmonic-to-noise ratio.

8. The method of claim 1, wherein the voiceprint received from the subject is recorded by the subject, or is provided via a live telecommunications connection.

9. The method of claim 1, wherein the subject is prompted to provide the voiceprint after a blood glucose level obtained from the subject, using a blood glucose monitor, is determined to be outside the predetermined acceptable blood glucose level range for the subject.

10. The method of claim 1, further comprising the step of receiving one or more contextual inputs from the subject, wherein the predefined correlation model further utilizes the received one or more contextual inputs from the subject to correlate the received voiceprint to a determined blood glucose level for the subject.

11. The method of claim 10, wherein the one or more contextual inputs comprise one or more of an emotional state of the subject, a psychological state of the subject, health of the subject, an activity of the subject, eating or drinking information, and sleep quality.

12. The method of claim 1, further comprising:
analyzing, with a trained compliance model, information about the subject to determine whether the subject is non-compliant or likely to be non-compliant with regard to providing voiceprints and/or blood glucose levels; and
prompting, when the trained compliance model determines that the subject is non-compliant or likely to be non-compliant, the subject to provide the voiceprint.

13. A system for monitoring a subject’s blood glucose level, comprising:
a predefined correlation model configured to correlate a voiceprint received from the subject to a determined blood glucose level for the subject;
a processor configured to: (i) receive a voiceprint from the subject; (ii) correlate the received voiceprint to a determined blood glucose level for the subject, using the predefined correlation model; (iii) determine, by comparing the determined blood glucose level to a predetermined acceptable blood glucose level range for the subject, whether the determined blood glucose level is outside the predetermined acceptable blood glucose level range for the subject; and (iv) generate an alert when the determined blood glucose level is outside the predetermined acceptable blood glucose level range for the subject.

14. The system of claim 13, wherein the predefined correlation model is a trained machine learning model, the trained machine learning model trained with at least one of: (1) a plurality of population-level measurements of blood glucose levels and corresponding voiceprints; and (2) a plurality of subject-specific measurements of blood glucose levels and corresponding voiceprints.

15. The system of claim 13, further comprising:
a blood glucose monitor configured to obtain one or more blood glucose levels from the subject; and
an audio sensor configured to obtain the voiceprint from the subject.
